(19) United States
(12) Patent Application Publication
(10) Pub. No.: US 2021/0330866 A1
(43) Pub. Date: Oct. 28, 2021

(54) Folded Porous Ingrowth Features for Medical Implants

(71) Applicant: Arthrex, Inc., Naples, FL (US)

(72) Inventors: Gary Scott Sherman, Naples, FL (US); Joel Zubizarreta, Naples, FL (US)

(21) Appl. No.: 17/270,604

(22) PCT Filed: Sep. 5, 2019
(86) PCT No.: PCT/US2019/049664

§ 371 Date: Feb. 23, 2021

Related U.S. Application Data
(60) Provisional application No. 62/727,219, filed on Sep. 5, 2018.

(51) Int. Cl. A61L 27/56; A61L 27/06; A61L 27/04; A61L 27/28; A61L 27/38; B33Y 80/00

(52) U.S. Cl. CPC A61L 27/56; A61L 27/06; A61L 27/045; A61L 2420/06; A61L 27/38; B33Y 80/00; A61L 2300/606; A61L 27/28

(57) ABSTRACT

The present disclosure provides folded, porous metal scaffolds that can be used as bone ingrowth features on medical implants.

### RELATED APPLICATIONS

This application claims the benefit of priority to U.S. Provisional Application No. 62/727,219 entitled “Folded Porous Ingrowth Features for Medical Implants,” filed on Sep. 5, 2018, the contents of which are hereby incorporated by reference in their entirety.

### BACKGROUND

Certain existing medical implant features do not allow for bone growth throughout the features of the implant as traditionally the features comprise a solid substrate coated with a porous substrate. Such a solid substrate does not allow for bone growth throughout the feature. Furthermore, such a solid substrate makes revision of the feature with a standard orthopedic saw blade difficult or impossible.

Accordingly, compositions and methods are disclosed herein that provide implantable medical devices having features that allow for better bone ingrowth and better revision characteristics.

### SUMMARY

The present disclosure relates to medical implants that are rigid, complex three-dimensional shapes that can be fixed to bone. As disclosed herein, folded, porous metal scaffolds allow for bone in-growth throughout the scaffold. The devices and methods described herein provide for contact of a rough surface of the folded, porous scaffold with bone. The scaffolds can also be cut by a standard orthopedic saw blade such that revision to a desired shape can be easily completed.

Thus, in a first aspect, the present disclosure provides a folded, metal, porous bone ingrowth device comprising one or more porous metal scaffolds having interconnected porosity and a rough side and a smooth side folded such that one or more portions of the rough side are present on an exposed surface of the device, one or more portions of the smooth side are in contact with each other and not exposed on a surface of the device, and one or more portions of the smooth side are exposed on a surface of the device. One or more portions of the smooth side can be exposed on a surface of the device and are configured to be attached to a medical implant. The one or more folded, metal, porous scaffolds can be one or more sheets having a thickness of about 0.5 mm prior to folding. The one or more porous metal scaffolds can have interconnected porosity between about 175 μm to about 300 μm, a mean porosity between about 50% to about 69%, and a plurality of pores each having a diameter ranging from about 400 μm to about 700 μm. Two or more of the one or more porous metal scaffolds can be affixed to one another. The folded, metal, porous bone ingrowth device can be coated with one or more biological materials, such as stem cells, bone marrow concentrate, platelet-rich plasma (PRP), a tissue graft particulate bone, or combinations thereof. The one or more porous metal scaffolds can comprise an open-celled metal sheet scaffold. The rough side of the one or more porous metal scaffolds can comprise a plurality of raised portions of material. The plurality of raised portions of material forming the rough side of the device can have a hardness greater than cortical bone. The plurality of raised portions of material forming the rough side of the device can have peripheral edges defining one or more curvatures forming curved peripheral surfaces. The plurality of raised portions of material forming the rough side of the device can have peripheral edges defining linear angles. The plurality of raised portions of material forming the rough side of the device can have a thickness ranging from about 100 μm to about 1,000 μm

In a second aspect, the present disclosure provides a medical device comprising a medical implant and a folded, porous, metal scaffold ingrowth device attached to the medical implant. The folded, metal, porous bone ingrowth device can be coated with one or more biological materials.

In a third aspect, the present disclosure provides a method of making a folded, metal, porous bone ingrowth device. The method comprises folding one or more porous metal scaffolds or one or more scaffold layers having a rough side and a smooth side such that one or more portions of the rough side are present on an exposed surface of the device, one or more portions of smooth side are in contact with each other and not exposed on a surface of the device, and one or more portions of the smooth side are exposed on a surface of the device. The folded, metal, porous bone ingrowth device can be attached to a medical implant or can be coated with one or more biological materials, or a combination thereof.

These as well as other aspects, advantages, and alternatives, will become apparent to those of ordinary skill in the art by reading the following detailed description, with reference where appropriate to the accompanying drawings.

### BRIEF DESCRIPTION OF THE DRAWINGS

FIG. 1A illustrates a single fin folded porous metal scaffold, according to an example embodiment.

FIG. 1B illustrates the porous metal scaffold of FIG. 1A prior to folding with the fold lines shown as dashed lines, according to an example embodiment.

FIG. 2A illustrates another porous metal scaffold prior to folding with the fold lines shown as dashed lines and the cut lines shown as solid lines, according to an example embodiment.

FIG. 2B another porous metal scaffold prior to folding with the fold lines shown as dashed lines, according to an example embodiment.

FIG. 3 illustrates a “Y” shaped folded, porous metal scaffold formed from the porous metal scaffolds of FIGS. 2A and 2B attached to a keel, according to an example embodiment.

FIG. 4A illustrates a double fin folded porous metal scaffold, according to an example embodiment.

FIG. 4B illustrates the porous metal scaffold of FIG. 4A prior to folding with the fold lines shown as dashed lines, according to an example embodiment.

FIG. 5A illustrates a tibial implant where the “T” shape is fashioned of folded, porous metal scaffolds, according to an example embodiment.

FIG. 5B illustrates a femoral implant where the “T” shapes and tubular shapes are fashioned of folded, porous metal scaffolds, according to an example embodiment.

FIG. 6 illustrates a “T” shaped scaffold attached to a wedge, according to an example embodiment.

### DETAILED DESCRIPTION

### A. Porous Metal Scaffold

Disclosed are medical implants that comprise one or more porous, rigid, features. The porous, rigid features can have complex three-dimensional shapes. These features help the medical device fixate to bone and allow for bone in-growth throughout the feature. The devices and methods provide for contact of a rough surface of the features with bone.

The device described herein may include one or more porous metal scaffolds. The one or more porous metal scaffolds may each comprise an open-celled metal sheet scaffold. The one or more porous metal scaffolds can have a mean porosity of about 50, 51, 52, 53, 54, 55, 56, 57, 58, 58.8, 59, 60, 61, 62, 63, 64, 65, 66, 67, 68, 69% or more (or any range between about 50 and 69%). Pore sizes can range from about 200 to about 700 μm (microns) in diameter. For example, pore sizes can be about 200, 250, 300, 350, 400, 425, 450, 475, 500, 523, 525, 550, 575, 600, 625, 650, 675, or 700 μM (or any range between about 200 and 700 μm). The sheets of porous metal scaffold can be about 0.25, 0.5, 0.75, 1.0, or 1.25 mm thick (or any range between about 0.25 and 1.0 mm). The one or more porous metal scaffolds may have interconnected porosity. The pore interconnectivity can be about 175, 200, 225, 229, 250, 275, or 300 μm (or any range between about 175 and 300 μm).

The one or more porous metal scaffolds can be manufactured by a layered approach. Individual layers of a metal, for example titanium, are etched to remove material. The layers can be about 0.1, 0.2, 0.25, 0.3, 0.4, 0.5 mm or more thick. The layers (e.g., 2, 3, 4, 5, or more layers) are diffusion bonded together to create interconnected porosity.

Where the one or more porous metal scaffolds are layered or stacked, each scaffold can have the same or different pore patterns. A pore pattern can comprise any number of pores and any type of pore shape.

When the pore pattern and size are the same, the layers can be stacked so that all pores align in the layers. Alternatively, the pores in one layer can overlap with pores of one or more other layers. The pores can extend through multiple layers and will have a shape defined by the overlap of pores.

When the pore pattern (i.e., number and shape of pores) of each layer is different, the pores in one layer will overlap with pores of one or more other layers. The pores can extend through multiple layers and will have a shape defined by the overlap of pores.

Therefore, a single porous metal scaffold can comprise multiple porous layers (e.g. 2, 3, 4, 5, 6, 7, 8, 9, 10, 15, 20, or more) that are bonded together.

In an embodiment The one or more porous metal scaffolds are not a 3D-printed scaffold, vapor deposition scaffold, or a spray-coated scaffold (e.g. a titanium plasma spray coated scaffold).

A given scaffold of the one or more porous metal scaffolds, whether bonded layers or a single layer, can include a rough surface or side and a smooth surface or side, as discussed in additional detail below. The top surface or side of the porous scaffold can have a texture, thereby making that side or surface rough. The bottom surface or side of the porous scaffold lacks a texture, making it smooth. The rough side of the porous, metal scaffold encourages bony ingrowth and therefore creates a stronger bond of the scaffold to the bone. The smooth side allows higher surface contact with a portion of a medical implant, which in turn gives a better bond strength between the porous scaffold and the implant.

The texture on the top surface or side of the porous scaffold can be, for example, raised portions of material (e.g., a metal, such as titanium, titanium alloy (e.g., Ti 6AL-4V, nitinol), cobalt chrome alloy, niobium, tantalum, or combinations thereof) that can be formed on or attached to the top surface or side of the porous scaffold. Unlike the layer material surrounding and defining the pores, the raised portions are disconnected from each other and provide raised shearing surfaces. In an embodiment, the raised portions of material have a hardness greater than cortical bone. A raised portion can be of any shape and can comprise a multitude of different shapes (e.g., circles, ovals, crescents, squares, rectangles, triangles, and free-form curved shapes). The distribution of the raised portions can be random or can have a pre-determined pattern. Each raised portion can be formed as a thickened portion of the top porous scaffold surface or side, which does not overlap with the pores formed in the top surface or side of a porous metal scaffold.

A raised portion can have peripheral surfaces defining one or more curvatures forming curved peripheral surfaces that apply shearing force to the bone as an scaffold is pressed against bone, and that can also direct the sheared material toward the pores of the porous material. A raised portion can also have peripheral surfaces that define linear angles, i.e., are flat. A raised portion can also have beveled edge. The raised portions do not cover an entirety of the material surrounding and defining the pores but only cover a portion of the material. The individual raised portions can have different thicknesses to form the rough side of a porous metal scaffold.

The raised portions can be formed in a separate material layer attached to the surface of the porous metal scaffold or can be formed as an integral part of the rough surface of the porous metal scaffold. For example, the raised portions can be formed from a layer of raised portion material that is bonded to the rough surface of the porous metal scaffold. The raised portions can also be made by additive manufacturing.

The texture of the rough surface of the one or more porous metal scaffolds, whether formed of raised portions or otherwise (e.g., scratching the surface), can be imparted to the porous metal scaffold in any suitable fashion. In an embodiment the thickness of the raised thickness of the raised portions can be about 100, 150, 200, 300, 400, 500, 600, 700, 800, 900, 1,000 microns or greater. In an embodiment the raised portions can be formed to have no spatial dimension, i.e., width, thickness, or length that is greater than about 100, 200, 300, 400, 500, 600, 700, 800, 900, or 1,000 microns.

In an embodiment, the raised portions are connected to the top surface material of the scaffold to form the texture, but, the raised portions can overlap with pores formed in the porous metal scaffold. In such an embodiment, the raised portions not only increase thicknesses of the scaffold material surrounding and defining the pores, but shear bone material, and direct the sheared bone material into the pores of the porous metal scaffold.

The one or more porous metal scaffolds can comprise a metal, such as titanium, titanium alloy (e.g., Ti 6AL-4V, nitinol), cobalt chrome alloy, niobium, tantalum, or combinations thereof. The one or more porous metal scaffolds can attach readily to metals (e.g., Ti, CoCr, and others), polymers (e.g., ultra-high molecular weight polyethylene (“UHMWPE”), polycarbonate-urethane (“PCU”), polyether ether ketone (“PEEK”), ceramics, and other materials used in medical devices.

The one or more porous metal scaffolds have a high coefficient of friction, for example, about 0.80, 0.85, 0.90, 0.95, 1.00, 1.05, 1.10, 1.20μ or more (or any range between about 0.80 and about 1.20μ). The one or more porous metal scaffolds have a structural stiffness of about 2.0, 2.5, 3.0, 3.2, 3.5, 4.0 k or more (or any range between about 2.0 and about 4.0 k).

In an embodiment, the one or more porous metal scaffolds have greater shear strength than PEEK, allograft, and titanium plasma spray coated implants. In an embodiment The one or more porous metal scaffolds have a shear strength of about 7, 8, 9, 10, 11, 12 MPa or more (or any range between about 7 and 12 MPa) in, for example, a porcine calvaria pin removal model at 5 weeks. In an embodiment, The one or more porous metal scaffolds have about 1.5, 1.8, 2.0, 2.5, 3.0, 3.3, 3.5, 4.0, 4.5, 5.0, 6.0, 6.5, 6.8, or more greater shear strength than materials such as PEEK, allograft, or titanium plasma spray coated implants.

The one or more porous metal scaffolds can be a BioSync®, OsteoSync™, Forticore®, InTice™, or OsteoFuZe® scaffold, as non-limiting examples.

### B. Folded Porous Metal Scaffold

The porous, folded metal scaffolds described herein have several advantages over other ingrowth features or implants including, for example, three-dimensionally printed, vapor deposition, or spray-coated implants. Three dimensionally printed, vapor deposition, or spray coated implants lose pores or pore structure upon folding and are not as strong and rigid as the instant devices and features. Folded, porous, metal scaffolds as described herein, however, retain their pores and pore structure upon folding and are strong and rigid.

The one or more porous metal scaffolds can be folded to form rigid complex three-dimensional shapes that can be used as a bone ingrowth device or feature. A scaffold can be folded and sintered in ways improve rigidity and functionality of the final construct, whether it comprises solely a folded, porous scaffold or one or more folded, porous scaffolds attached to medical device. A bone ingrowth device can comprise one or more metal porous scaffolds having a rough side and a smooth side folded such that one or more portions of the rough side are present on an exposed surface of the device, one or more portions of smooth side are in contact with each other and not exposed on a surface of the device, and one or more portions of the smooth side are exposed on a surface of the device. In general, most or all of the rough side is on an exposed surface of the completed folded porous metal scaffold. That is about 55, 60, 70, 75, 80, 90, 95, or 100% of the rough side of the folded, porous metal scaffold is exposed on the surface of the completed device. This provides surface area for bone to affix to and then in-grow. In general, most or all of the smooth side of the porous metal scaffold is not exposed to the external surface of the folded metal scaffold. One or more portions of the smooth side of the folded porous metal scaffold can be in contact with one another after folding.

In an embodiment, one or more portions of the smooth side of the folded, porous metal scaffold exposed on a surface of the device are attached to an implant, such as a medical implant.

The scaffold is folded such that most or all of the rough side is on the exterior and exposed to the body of a subject. The shapes and roughness of the folded, metal porous scaffold provide an initial fixation on bone and provide for bone ingrowth into the folded, porous metal scaffold. Because the folded, porous metal scaffold has interconnected porosity and no solid supporting substrate, bone can grow completely through the scaffold.

Two or more porous metal scaffolds can be joined together and then folded. Alternatively, two or more porous metal scaffolds can be folded and then joined together. In an embodiment, two or more porous metal scaffolds (e.g., 2, 3, 4, 5 or more) can be stacked together to form a layered scaffold and then folded. A layered scaffold is layered or stacked such that one surface (e.g. top surface) is rough and another surface (e.g., bottom surface) is smooth.

A folded, porous metal scaffold (and final implant construct) has superior bone ingrowth performance. For example, about 60, 70, 80, 90% or more of the void space of the porous metal scaffold can be occupied by bone at about 6, 7, 8, 9, 10, 11, 12 or more weeks after implantation into a subject (e.g., a mammal, such as a human). In an embodiment about 75%, 80%, 85%, 90%, 95% or more of the void space is occupied by bone at 6 weeks. In an embodiment about 90% of the void space is occupied by bone at 6, 8, 11, 12, or 24 weeks after implantation.

A folded, porous metal scaffold (and final implant construct) has low debris generation during insertion.

In an embodiment, a porous metal scaffold (and final implant construct) has about 5, 4, 3, 2, 1, 0.1 or less abrasion (that is about 5, 4, 3, 2, 1, 0.1 or less percent loss after multiple insertion cycles. Other types of porous implants, for example titanium plasma spray and sintered bead implants have significantly more abrasion after 10 insertion cycles (e.g., about 5, 10, 20, 30, 40% or more mass loss).

Advantageously, since the folded, porous metal scaffold has no solid supporting substrate, it can be cut through with a standard orthopedic saw blade if need for revision arises.

In an embodiment a device comprising a folded, porous metal scaffold is coated, soaked, or contacted with one or more biological materials prior to implantation. Biological materials can include, for example, stem cells, bone marrow concentrate, platelet-rich plasma (PRP), a tissue graft, particulate bone, and combinations thereof.

### C. Shapes of the Folded Porous Metal Scaffold

As described above, a given scaffold of the one or more porous metal scaffolds described herein can include a rough surface or side and a smooth surface or side. The top surface or side of the porous scaffold can have a texture, thereby making that side or surface rough. The bottom surface or side of the porous scaffold lacks a texture, making it smooth. The rough side of the porous, metal scaffold encourages bony ingrowth and therefore creates a stronger bond of the scaffold to the bone. The smooth side allows higher surface contact with a portion of a medical implant, which in turn gives a better bond strength between the porous scaffold and the implant. In each of the embodiments described herein with respect to the Figures, one or more portions of the smooth side are not exposed on the surface of the folded, porous metal scaffold and are within the folded structure. In addition, one or more portions of the smooth side of the folded, porous metal scaffold can be exposed on the surface. The exposed smooth side can be used to affix the folded, porous metal scaffold to a medical implant, while the exposed rough side can be used to affix the folded, porous metal scaffold to bone.

The one or more porous metal scaffolds can be folded into any desired shape, including “I,” “U,” “T,” “S,” “O”, “C”, “D”, “E”, “F”, “H”, “J”, “L”, “V”, “W,” “X,” or “Z” shapes. Other shapes include a single fin or double fin shape, tubular shapes, and wedge shapes. A shape can be designed to allow for strong initial fixation to bone and to resist anatomical loading.

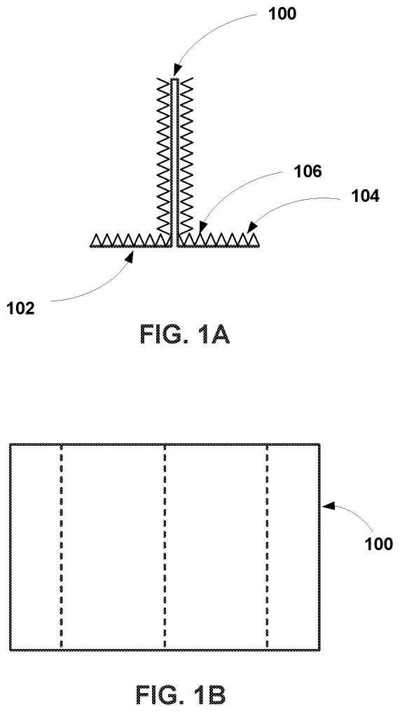

With reference to the Figures, a single fin as shown in FIG. 1A can be made by folding a porous metal scaffold 100 as shown in FIG. 1B, where the dashed lines represent the fold lines. The smooth side 102 and rough side 104 of the porous metal scaffolding 100 are shown in FIG. 1A. FIG. 1A further illustrates the raised portions 106 defining the rough side 104 of the porous metal scaffold 100.

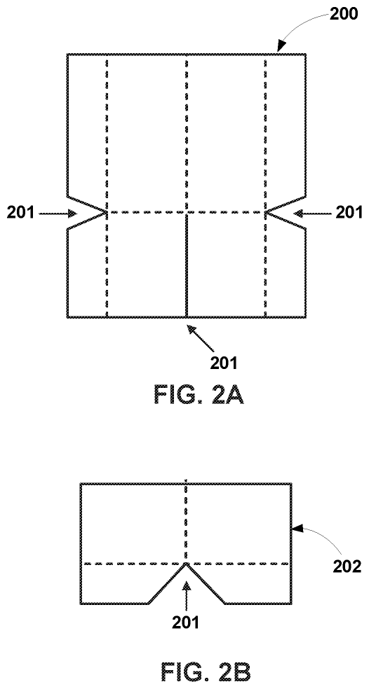

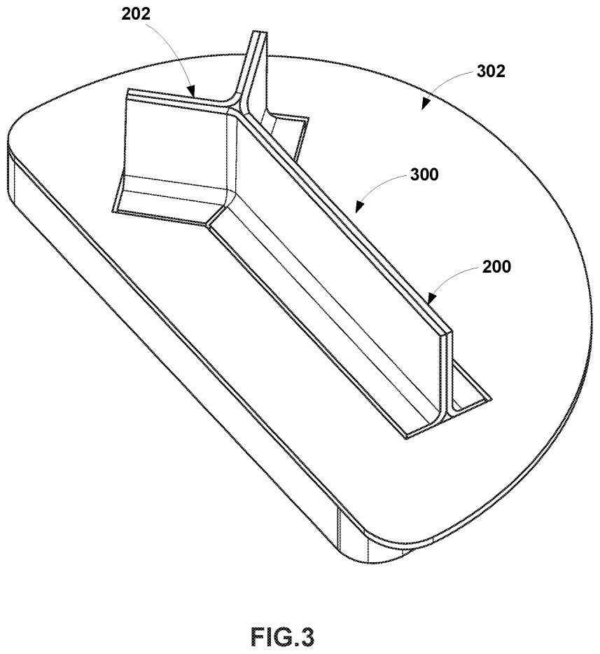

Two porous metal scaffolds (FIGS. 2A-2B) can be folded and attached to one another to form “Y” shape as shown in FIG. 3. A first porous metal scaffold 200 (FIG. 2A) can folded along the dashed lines and cut at line 201. A second porous metal scaffold 202 (FIG. 2B) can be folded along the dashed lines and then joined together to form the “Y” shape 300 shown in FIG. 3. As shown in FIG. 3, the combined first porous metal scaffold 200 and second porous metal scaffold 202 may be attached to a keel 302. Such a keel 302 can be used for resistance to anterior shear forces.

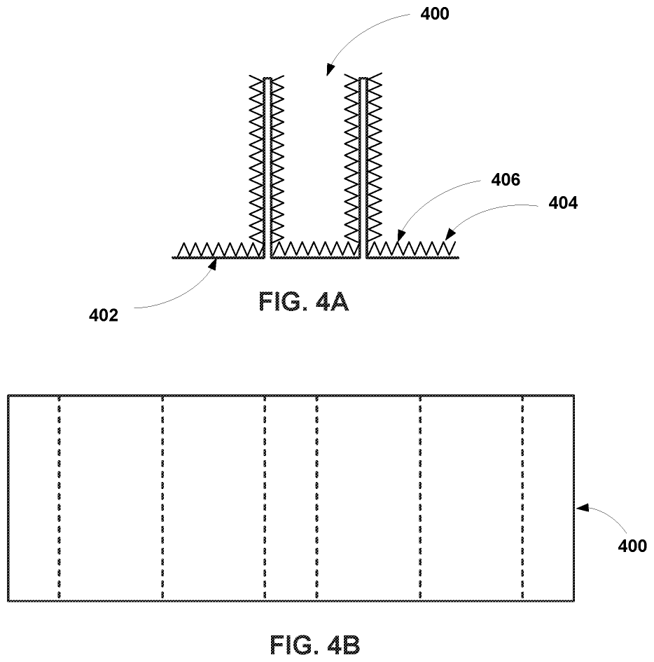

In another example as shown in FIG. 4, a porous metal scaffold 400 can be folded into a double fin shape. In particular, the porous metal scaffold 400 can be folded along the dashed lines as shown in FIG. 4B to form the double fin shape illustrated in FIG. 4A. The smooth side 402 and rough side 404 of the porous metal scaffold 400 are shown in FIG. 4A. FIG. 4A further illustrates the raised portions 406 defining the rough side 404 of the porous metal scaffold 400.

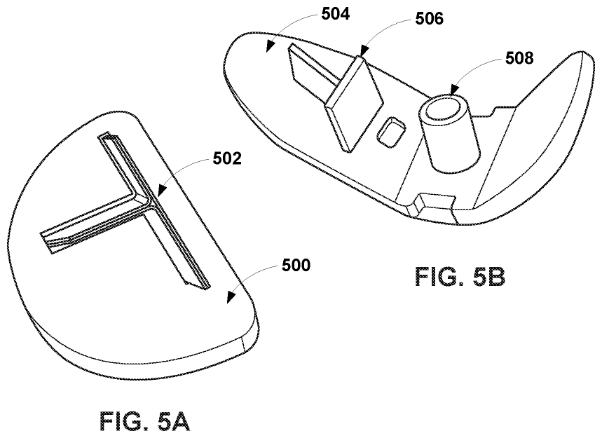

FIG. 5A illustrates a tibial implant 500 where the “T” shape is fashioned of folded, porous metal scaffolds 502. As shown in FIG. 5A, the “T” shaped folded, porous metal scaffolds 502 are coupled to the tibial implant 500. In one example, a smooth side of the “T” shaped folded, porous metal scaffolds 502 is coupled to the tibial implant 500, while a rough side of the “r” shaped folded, porous metal scaffolds 502 forms the exterior surface of the “T” shape.

FIG. 5B illustrates a femoral implant 504 where the “T” shape 506 and tubular shape 508 are fashioned of folded, porous metal scaffolds. In one example, a smooth side of the “T” shaped folded, porous metal scaffolds 506 is coupled to the femoral implant 504, while a rough side of the “T” shaped folded, porous metal scaffolds 506 forms the exterior surface of the “T” shape. Similarly, in one example, a smooth side of the tubular shape 508 is coupled to the femoral implant 504, while a rough side of the tubular shape 508 forms the exterior surface of the tubular shape.

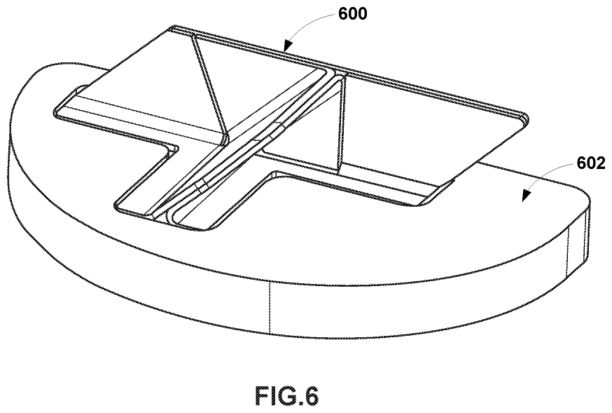

FIG. 6 illustrates a “T” shaped scaffold 600 attached to a wedge 602. In particular, FIG. 6 illustrates three porous metal scaffolds are folded and affixed to each other to form the “T” shape 600. The folded porous metal scaffolds can be attached to, for example, a solid titanium alloy wedge or a solid titanium alloy wedge that is covered with about 0.25 mm, 0.5 mm, or 1 mm of porous metal scaffold.

### D. Medical Implant

One or more portions of a folded, porous metal scaffold can be affixed to an implant, such as a medical implant. In an embodiment, one or more smooth portions of the folded metal scaffold are affixed to an implant. A medical implant can be, for example, a unicompartmental knee implant, knee tibial implant, knee femoral implant, total knee implant, total hip implant, ankle implant, shoulder implant, elbow implant, wrist implant, spinal implant, or cervical implant.

A medical implant can be made of any material including, for example, metal (e.g., stainless steel, cobalt-chromium alloys, titanium, titanium alloys, tantalum, zirconium alloys, oxinium oxidized zirconium, and others), polymeric materials (e.g., polyethylene (such as ultra-high cross linked polyethylene (UHXLPE) or ultra-high molecular weight polyethylene (UHMWPE)), polyvinylidene fluoride, polypropylene, polydimethylsiloxane, parylene polyamide, polytetrafluoroethylene, poly(methylmethacrylate), polyamide, polyurethane), ceramics (e.g., silicates, metallic oxides, carbides, refractory hydrides, refractory sulfides, and refractory selenides), other material suitable for medical implants, or combinations thereof.

One or more folded metal scaffolds can be attached to a medical implant by any method known in the art, for example, sintering, welding, bonding, fastening, and other methods.

### E. Methods

Methods of making the folded, metal, porous bone ingrowth device of any one of the embodiments are described above. In particular, a method comprises folding one or more metal porous scaffolds, or one or more scaffold layers, having a rough side and a smooth side such that one or more portions of the rough side are present on an exposed surface of the device, one or more portions of smooth side are in contact with each other and not exposed on a surface of the device, and one or more portions of the smooth side are exposed on a surface of the device. A method may also include attaching the folded, metal, porous bone ingrowth device to a medical implant, as discussed above. A method may also include coating the folded, metal, porous bone ingrowth device with one or more biological materials.

### F. Examples

The following example is for exemplification purposes only and is not intended to limit the scope of the invention described in broad terms above.

A commercially pure titanium metal scaffold with a porosity of about 60%, a mean pore size of about 434-660 μm, pore interconnectivity of about 229 μm, a nominal thickness of about 1 mm (a range of about 5 mm to 1 mm) was used to construct a bone ingrowth feature. A first and second porous metal scaffold were cut and folded as shown in FIG. 2A-2B. The dashed lines are fold lines. Cut lines are shown at 201. The two porous metal scaffolds were folded and attached to each other to form a “Y” shaped folded, porous metal scaffold shown in FIG. 3.

Exemplary devices and methods are described herein. It should be understood that the word “exemplary” is used herein to mean “serving as an example, instance, or illustration.” Any embodiment or feature described herein as “exemplary” is not necessarily to be construed as preferred or advantageous over other embodiments or features. The exemplary embodiments described herein are not meant to be limiting. One of ordinary skill in the art will readily understand that certain aspects of the disclosed systems and methods can be arranged and combined in a wide variety of different configurations, all of which are contemplated herein. The terms used in the specification generally have their ordinary meanings in the art, within the context of the compositions and methods described herein, and in the specific context where each term is used.

Furthermore, the particular arrangements shown in the Figures should not be viewed as limiting. It should be understood that other embodiments may include more or less of each element shown in a given Figure. Further, some of the illustrated elements may be combined or omitted. Yet further, an exemplary embodiment may include elements that are not illustrated in the Figures.

As used herein, with respect to measurements, “about” means +/−5%. For example, for a value of about 100, means 95 to 105 (or any value between 95 and 105).

Whenever a range is given in the specification, for example, a size range, a time range, or a concentration range, all intermediate ranges and subranges, as well as all individual values included in the ranges given are intended to be included in the disclosure. It will be understood that any subranges or individual values in a range or subrange that are included in the description herein can be excluded from the aspects herein.

In addition, where features or aspects of the invention are described in terms of Markush groups or other grouping of alternatives, those skilled in the art will recognize that the invention is also thereby described in terms of any individual member or subgroup of members of the Markush group or other group.

As used herein, “coupled” means associated directly as well as indirectly. For example, a member A may be directly associated with a member B, or may be indirectly associated therewith, e.g., via another member C. It will be understood that not all relationships among the various disclosed elements are necessarily represented.

Unless otherwise indicated, the terms “first,” “second.” etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item. As used in the description herein and throughout the claims that follow, the meaning of “a”, “an”, and “the” includes plural reference unless the context clearly dictates otherwise.

Reference herein to “one embodiment” or “one example” means that one or more feature, structure, or characteristic described in connection with the example is included in at least one implementation. The phrases “one embodiment” or “one example” in various places in the specification may or may not be referring to the same example.

As used herein, a system, apparatus, device, structure, article, element, component, or hardware “configured to” perform a specified function is indeed capable of performing the specified function without any alteration, rather than merely having potential to perform the specified function after further modification. In other words, the system, apparatus, device, structure, article, element, component, or hardware “configured to” perform a specified function is specifically selected, created, implemented, utilized, programmed, and/or designed for the purpose of performing the specified function. As used herein, “configured to” denotes existing characteristics of a system, apparatus, device, structure, article, element, component, or hardware which enable the system, apparatus, device, structure, article, element, component, or hardware to perform the specified function without further modification. For purposes of this disclosure, a system, apparatus, device, structure, article, element, component, or hardware described as being “configured to” perform a particular function may additionally or alternatively be described as being “adapted to” and/or as being “operative to” perform that function.

It will be appreciated that other arrangements are possible as well, including some arrangements that involve more or fewer steps than those described above, or steps in a different order than those described above.

While various aspects and embodiments have been disclosed herein, other aspects and embodiments will be apparent to those skilled in the art. All embodiments within and between different aspects of the invention can be combined unless the context clearly dictates otherwise. The various aspects and embodiments disclosed herein are for purposes of illustration and are not intended to be limiting, with the true scope and spirit being indicated by the claims.

1. A folded, metal, porous bone ingrowth device comprising:
one or more porous metal scaffolds having interconnected porosity and a rough side and a smooth side folded such that one or more portions of the rough side are present on an exposed surface of the device, one or more portions of the smooth side are in contact with each other and not exposed on a surface of the device, and one or more portions of the smooth side are exposed on a surface of the device.

2. The device of claim 1, wherein the one or more portions of the smooth side that are exposed on the surface of the device are configured to be attached to a medical implant.

3. The device of claim 1, wherein the one or more porous metal scaffolds comprise one or more sheets having a thickness of about 0.5 mm prior to folding.

4. The device of claim 1, wherein the one or more porous metal scaffolds have interconnected porosity of between about 175 μm to about 300 μm.

5. The device of claim 1, wherein a mean porosity of the one or more porous metal scaffolds is between about 50% to about 69%.

6. The device of claim 1, wherein the one or more porous metal scaffolds include a plurality of pores each having a diameter ranging from about 400 μm to about 700 μm.

7. The device of claim 1, wherein two or more of the one or more porous metal scaffolds are affixed to one another.

8. The device of claim 1, wherein the folded, metal, porous bone ingrowth device is coated with one or more biological materials.

9. The device of claim 8, wherein the biological materials comprise stem cells, bone marrow concentrate, platelet-rich plasma (PRP), a tissue graft, particulate bone, or combinations thereof.

10. The device of claim 1, wherein the one or more porous metal scaffolds comprise an open-celled metal sheet scaffold.

11. The device of claim 1, wherein the rough side of the one or more porous metal scaffolds comprise a plurality of raised portions of material.

12. The device of claim 11, wherein the plurality of raised portions of material forming the rough side of the device have a hardness greater than cortical bone.

13. The device of claim 11, wherein the plurality of raised portions of material forming the rough side of the device have peripheral edges defining one or more curvatures forming curved peripheral surfaces.

14. The device of claim 11, wherein the plurality of raised portions of material forming the rough side of the device have peripheral edges defining linear angles.

15. The device of claim 11, wherein the plurality of raised portions of material forming the rough side of the device have a thickness ranging from about 100 μm to about 1,000 μm.

16. A medical device comprising:
(a) a medical implant; and
(b) a folded, porous, metal scaffold ingrowth device of claim 1 attached to the medical implant.

17. The medical device of claim 16, wherein the folded, metal, porous bone ingrowth device is coated with one or more biological materials.

18. A method of making the folded, metal, porous bone ingrowth device of claim 1, comprising:
folding one or more porous metal scaffolds or one or more scaffold layers having a rough side and a smooth side such that one or more portions of the rough side are present on an exposed surface of the device, one or more portions of smooth side are in contact with each other and not exposed on a surface of the device, and one or more portions of the smooth side are exposed on a surface of the device.

19. The method of claim 18, further comprising attaching the folded, metal, porous bone ingrowth device to a medical implant.

20. The method of claim 18, further comprising coating the folded, metal, porous bone ingrowth device with one or more biological materials.
